(19) United States
(12) Patent Application Publication
(10) Pub. No.: US 2006/0122101 A1
(43) Pub. Date: Jun. 8, 2006

(54) Drug for preventing, regulating or treating adhesion

(76) Inventors: Mizuo Miyazaki, Kyoto (JP); Shinji Takai, Osaka (JP)

Correspondence Address: KILYK & BOWERSOX, P.L.L.C., 400 HOLIDAY COURT, SUITE 102, WARRENTON, VA, 20186, US

(21) Appl. No.: 10/544,254

(22) PCT Filed: Feb. 4, 2004
(86) PCT No.: PCT/JP04/01111

§ 371 Date: Aug. 23, 2005

(30) Foreign Application Priority Data
Feb. 5, 2003 (JP) 2003-028743

(51) Int. Cl. A61K 38/05

(52) U.S. Cl. 514/7

(57) ABSTRACT

A problem of the present invention is to provide a medicament which allows effective prevention, inhibition, or treatment of adhesion formation, comprising using before or after a variety of surgical treatments including orthopedic or plastic surgery, for example, in cardiac, thoracic, gynecological, ophthalmic, abdominal sites or in the case where injury or inflammation can cause adhesion in visceral organs. The medicament contains an effective amount of at least one protease inhibitor to administer intravenously, orally, or percutaneously. The protease inhibitor is preferably a serine protease inhibitor, and the serine protease inhibitor is a chymotrypsin-like serine protease inhibitor. Specifically, it is a chymase inhibitor, the peptide derivative of an aryl diester of alpha-aminoalkylphosphonic acid, Suc-Val-Pro-Phe^{P}(OPh)₂, and preferably the enantiomer, Suc-Val-Pro-L-Phe^{P}(OPh)₂.

The present application claims the priority based on Japanese Patent Application No. 2003-028743 which is incorporated herein for reference.

### TECHNICAL FIELD OF THE INVENTION

The present invention relates to a method for preventing adhesion formation using protease inhibitors. More specifically, this invention relates to a method for preventing adhesion formation using a serine protease inhibitor, particularly a chymase inhibitor.

### BACKGROUND OF THE INVENTION

Postoperative adhesion formation is a major complication occurring following, for example, a general orthopedic or plastic surgery for cardiac, thoracic, gynecologic, ophthalmic and abdominal sites. Pericardial adhesions pose a significant problem and increase morbidity and mortality of reoperative cardiac surgical procedures (J. Invest. Surg. 14: 93-97, 2001: Non-patent document 1). Postoperative peritoneal adhesion is one of the major causes of diseases' susceptibility accompanying with complications such as intestinal obstruction, infertility, and pain in the serious cases after abdominal or gynecologic surgery (Dig. Surg. 18: 260-73, 2001: Non-patent document 2). While a number of causing factors have been implicated in adhesion formation including trauma, desiccation, local ischemia, peritonitis of infectious origins and endometriosis, the pathophysiological nature of adhesion formation is not completely understood.

Postoperative adhesions generally occur as a result of the normal wound healing response within the fifth to seventh day after injury. It is considered that adhesion formation and adhesion-free re-epithelialization are alternative pathways, both of which begin with coagulation, and which initiate a cascade of events resulting in the buildup of fibrin gel matrix. In the case where fibrin deposition is in excess or not removed, the fibrins crosslink to form the fibrin gel matrix, which may then serve as a progenitor to adhesions. The crosslink works with cellular elements such as fibroblast to give a basis for adhesion tissue. In contrast to this, a protective system for fibrinolytic enzyme in perineum such as the plasmin system can remove the fibrin gel matrix. However, surgery dramatically attenuates fibrinolytic activity. Therefore, it is determined depending upon the degrees of the damage and fibrinolysis in the tissue surface which pathway of adhesion formation and re-epitheliazation is selected.

Although a variety of approaches toward the prevention and treatment of adhesion formation have been performed, these have resulted only in the limited fruits and the undesirable side-effects (The Peritoneum, Springer-Verlag, NY pp. 307-369, 1992; Non-patent document 3). For example, in an attempt to prevent fibrin deposition, peritoneal lavages to wash away fibrinous exudates, surgical techniques to minimize tissue ischemia, and utilization of barriers to limit adhesion of the treating surface of serous membrane have been tested. Alternatively, anti-inflammatory drugs such as corticosteroids and non-steroidals have been utilized (Clin. Exp. Gynecol. 28: 126-127, 2001: Non-patent document 4). A method for removing fibrin deposition by fibrinolysis using the conventional proteolytic enzyme following the skeletogenous stage is now on study (Int. Surg. 83:11-14, 1998: Non-patent document 5). It cannot be described that there has been demonstrated a universally effective method for preventing or treating formation of the postoperative adhesion.

Mast cells play an important role in induction of allergic inflammatory responses. Mast cells express receptors in the cell surfaces for binding the Fc portions of IgE and certain classes of IgG, allowing antibodies to bind to the surfaces. Interaction of antigens with the bound antibodies results in the release from mast cells of a series of potent mediators such as histamine, serotonin, cytokines and a variety of enzymes that play critical roles in coping with the initiation of allergic and anaphylactic-type responses. In addition, it has been also pointed out that mast cells are involved with peritoneal adhesion formation (Am. J. Surg. 165: 127-130, 1993: Non-patent document 6). It has been reported that mast cell stabilizers, which inhibit the activation and accumulation of mast cells, are effective in attenuating adhesion formation in rat models (Surg. Today 29: 51-54, 1999: Non-patent document 7). Moreover, it have been also demonstrated that adhesion formation in mast cell deficient mice was significantly less severe than that in normal control mice (J. Surg. Res. 92: 40-44, 2000: Non-patent document 8). These reports suggest that mast cells are closely related to adhesion formation. However, it has not yet been clarified what role is rightly played by mast cells and which of substances released from mast cells might be involved in the adhesion.

Chymase is a chymotrypsin-like serine protease contained in the secretory granules of mast cells. Chymase is a cytokine which activates stem cell factors and has an ability to induce the accumulation of mast cells. For example, it has been reported that chymase activity was significantly increased in treating sites after cecal scraping in mice (J. Surg. Res. 92: 40-44, 2000: Non-patent document 8). Specifically, it seems that a chymase positive mast cell plays a roll in the development of adhesion formation. However, the pathophysiological role of chymase in the development of adhesion formation remains unclear. In order to clarify the relationship between chymase and adhesion formation, we have now studied on protease inhibitors which have an effect against chymase, especially, on the possibility in effect of using them as means for preventing and inhibiting adhesion formation.

There has been a significant desire for a method for effectively preventing and inhibiting adhesion formation following a variety of surgical procedures including cardiac, thoracic, gynecologic, ophthalmic, and abdominal surgeries. For the prevention of adhesion, a method for promoting synthesis and/or secretion of plasminogen activator by mesothelial cell has been disclosed (Japanese Patent Publication No. 2002-506817 gazette: Patent document 1).

Moreover, we have now applied a patent in the United States of America for a method for preventing/inhibiting adhesion formation, re-formation using protease inhibitors. There are provided herein a method for preventing/inhibiting adhesion formation between tissue surfaces in a vertebrate subject, in order to prevent/inhibit the formation/re-formation of adhesion to the organs such as peritoneal or pleural cavity or the other body regions following surgical or accidental injury in mammals, comprising administering chymase inhibitors (for example: alpha-aminoalkylphosphonate derivatives) and the other serine protease inhibitors to the region of tissue surface of the subject for a sufficient period to permit repair of the tissue.

However, these methods, which are methods for administering the drug on the surface of the tissue with the administration period limited to a certain period following the surgical operations, are not sufficient to prevent and inhibit the adhesion formation. Furthermore, the conventional methods can not treat an adhesion which inflammation may cause within a body.

(Non-patent document 1) Krause et al., J. Invest. Surg. 14: 93-97, 2001

(Non-patent document 2) Liakakos et al., Dig. Surg. 18: 260-73, 2001

(Non-patent document 3) dizerega and Rogers, The Pertioneum {Peritoneum}, Springer-Verlag, NY pp. 307-369,

(Non-patent document 4) Baysal, Clin. Exp. Gynecol. 28: 126-127, 2001

(Non-patent document 5) Hioki et al., Int. Surg. 83: 11-14,

(Non-patent document 6) Liebman et al., Am. J. Surg. 165: 127-130, 1993

(Non-patent document 7) Adachi et al., Surg. Today 29: 51-54, 1999

(Non-patent document 8) Yao et al., J. Surg. Res. 92: 40-44,

(Patent document 1) Japanese Patent Publication No. 2002-506817 gazette

### DISCLOSURE OF THE INVENTION

An object of the present invention is to provide a medicament which allows effective prevention/inhibition of adhesion formation, comprising using before or after a variety of surgical treatments including orthopedic or plastic surgery, for example, in cardiac, thoracic, gynecological, ophthalmic, abdominal sites or in the case where injury or inflammation can cause adhesion in visceral organs.

The present inventors have taken note of the fact that a sufficient amount of at least one protease inhibitor is administered for a certain period for repair of a tissue where adhesion can be formed, allowing prevention of adhesion formation. They made a diligent study and, as a result, have found that a medicament containing the protease inhibitor can be administered intravenously, orally or percutaneously to solve the above-described problem, and completed the present invention.

Specifically, the present invention comprises:

1. A medicament for preventing, inhibiting, or treating adhesion formation of the tissue surface within a vertebrate subject, wherein the medicament contains an effective amount of at least one protease inhibitor and is administered intravenously, orally, or percutaneously,

2. The medicament for preventing, inhibiting or treating adhesion formation according to Item 1, wherein the protease inhibitor is a serine protease inhibitor,

3. The medicament for preventing, inhibiting or treating adhesion formation according to Item 2, wherein the serine protease inhibitor is a chymotrypsin-like serine protease inhibitor,

4. The medicament for preventing, inhibiting or treating adhesion formation according to Item 3, wherein the chymotrypsin-like serine protease inhibitor is a chymase inhibitor,

5. The medicament for preventing, inhibiting or treating adhesion formation according to Item 4, in which the relevant chymase inhibitor is a peptide derivative of aryl diester of alpha-aminoalkylphosphonic acid,

6. The medicament for preventing, inhibiting or treating adhesion formation according to Item 4, wherein the chymase inhibitor is Suc-Val-Pro-Phe^{P}(OPh)₂,

7. The medicament for preventing, inhibiting or treating adhesion formation according to Item 4, wherein the chymase inhibitor is a concentrated preparation of enantiomer Suc-Val-Pro-L-Phe^{P}(OPh)₂ of Suc-Val-Pro-Phe^{P}(OPh)₂,

8. The medicament for preventing, inhibiting or treating adhesion formation according to Item 7, wherein Suc-Val-Pro-L-Phe^{P}(OPh)₂ contains 95% or more of the total weight of Suc-Val-Pro-Phe^{P}(OPh)₂ in the concentrated preparation of the enantiomer,

9. The medicament for preventing, inhibiting or treating adhesion formation according to any one of Items 1-8, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellose, dextran, cyclodextran and a composition of compounds thereof,

10. A medicament for preventing, inhibiting or treating adhesion formation, wherein the medicament comprises the protease inhibitor according to any one of Items 1-9 and a pharmaceutically acceptable diluent solution or excipient, and

11. A method for preventing, inhibiting or treating healing formation, wherein the medicament for preventing, inhibiting or treating healing formation according to any one of Items 1-8 is administered to a vertebrate subject before surgical operation, during the surgical operation, after the surgical operation, or in the case of possible inflammatory visceral adhesion.

### BRIEF DESCRIPTION OF THE DRAWINGS

FIG. 1 is a graph showing the average adhesion score when a placebo or Suc-Val-Pro-L-Phe^{P}(OPh)₂ was orally administered (Experimental Example).

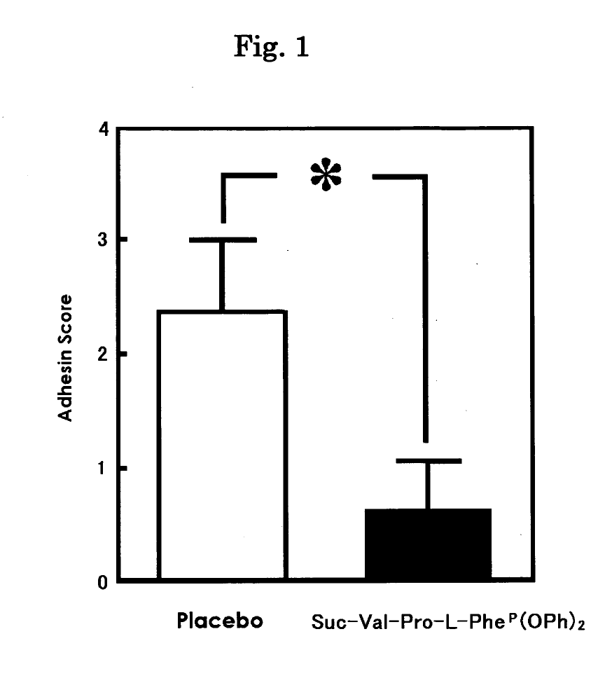

### DESCRIPTION OF THE PREFERRED EMBODIMENT(S)

The present invention relates to a medicament for preventing, inhibiting or treating adhesion formation of the tissue surface within a vertebral subject, wherein the medicament containing an effective amount of a protease inhibitor is administered intravenously, orally, or percutaneously. The protease inhibitor can be administered to the subject, for example, before/during/after the surgical treatment. Moreover, even in the other case than the surgical operation, for example, in the case of injury or possible inflammatory visceral adhesion, the inhibitor can be administered.

The medicament of the present invention is a medicament for preventing, inhibiting or treating adhesion formation in the abdominal cavity of a warm blooded mammal, and comprises administering intravenously, orally, or percutaneously an effective amount of at least one serine protease inhibitor to the mammal by a sufficient amount for a certain period to permit repair of the tissue. The preferred Embodiment relates to a method for preventing/inhibiting postoperative adhesion formation using at least one inhibitor against the serine protease, that is, a chymotrypsin-like serine protease. The medicament of the present invention can be applied to human as one of the warm blooded mammal.

(Protease Inhibitor)

The protease inhibitor contained in the medicament of the present invention is a known substance. Any protease inhibitor can be used regardless of a method used for preparing it, as long as it is purified to a necessary degree for pharmaceutical use.

The protease targeted by the medicament of the present invention for preventing, inhibiting or treating adhesion formation is preferably a serine protease. The serine protease is a subclass of endopeptidase which cleaves and binds serine in a peptide (Barrett, A. J., In: Protease Inhibitors, Ed. Barrett, A. J. et al., Elsevier, Amsterdam, pp. 3-22, 1986). Serine protease itself is known. For example, two super families of serine proteases, i.e., a chymotrypsin super family and a Streptomyces subtilisin super family have been reported.

The serine protease inhibitor is known, and can be classified into the following families: including (1) a family of trypsin inhibitor (Kunitz) derived from pancreas of bovine, which is also known as a basic protease inhibitor (Ketcham, L. K. et al, In: Atlas of Protein Sequence and Structure, Ed. Dayhoff, M. O., pp. 131-143, 1978) (hereinafter, abbreviated as “BPTI”), (2) Kazal family, (3) Streptomyces subtilisin inhibitor family (hereinafter, abbreviated as “SSI”), (4) the serpin family, (5) soybean trypsin inhibitor (Kunitz) family, (6) potato inhibitor family, (7) Bowman-Birk family (Laskowski, M. et al, Ann. Rev. Biochem., 49: pp. 593-626, 1980). Crystallographic data are available for a number of intact inhibitors including members of the BPTI, Kazal, SSI, soybean trypsin, potato trypsin families and for a cleaved form of the Seprin-alpha-1-antitrypsin cleavage type (Read, R. J. et al). Many of serine protease inhibitors have specificity for a broad range of protease families, and can inhibit both the chymotripsin superfamily of protease including blood coagulation serine protease and the Streptomyces subtilisin super family of serine protease (Laskowski et al, Ann. Rev. Biochem., 49: pp. 593-626, 1980). The specificity of each inhibitor is thought to be determined by the identity of the amino acid that is immediately amino-terminal to the site of potential cleavage of the inhibitor by the serine protease. This amino acid, known as P site residue, is thought to form an acyl bond with the serine in the active site of the serine protease (Laskowski et al, Ann. Rev. Biochem., 49: pp. 593-626, 1980).

The preferred serine protease inhibitor contained in the medicament of the present invention belongs to the serpin family and the Bowman-Birk family. The serine protease inhibitor against the serpin family includes plasminogen activator inhibitors PAI-1, PAI-2, and PAI-3, and C1 esterase-inhibitor, alpha-2-antiplasmin, contrapsin, alpha-1-antitrypsin, antithrombin III, protease nexin I, alpha-1-antichymotrypsin, protein C inhibitor, heparin cofactor II and growth hormone regulated protein (Carrell et al, Cold Spring Harbor Symp. Quant. Biol., 52: 527-535, 1987).

The examples of serine proteases of chymotrypsin superfamily include tissue-type plasminogen activator (t-PA), trypsin, trypsin-like protease, chymotrypsin, plasmin, elastase, urokinase (or non-uriary type plasminogen activator: u-PA), acrosin, activated protein C, C1 esterase, cathepsin G, chymase, and proteases of the blood coagulation cascade including kallikrein, thrombin, Factor VIIa, Factor IXa, Factor Xa, Factor XIa, Factor XIIa (Barrett, A. J., In: Protease Inhibitors, Ed. Barrett, A. J. et al., Elsevier, Amsterdam, pp. 3-22, 1986; Strassburger, W. et al., FEBS Lett., 157: pp. 219-223, 1983).

The catalytic domains in all the serine proteases of the chymotrypsin superfamily have homology in sequence and homology in structure. The homology in sequence comprises (1) a specific active site residue, for example, in the case of trypsin, serine located at 195, histidine at 57, and aspartic acid at 102 which are common; (2) an oxyanion hole (for example, glycine at 193 and aspartic acid at 194 in the case of trypsin), (3) a cysteine residue forming a structural disulfide crosslink, which are all reserved (Hartley, B. S., Symp. Soc. Gen. Micrbopl., 24: pp. 152-182 (1974)).

The homology in structure comprises (1) a common fold that consist of two Greek key structures (Richardson), (2) common factors of catalytic residue, (3) precise conservation of structure in a molecular nucleus (Stroud, R. M., Sci. AM., 231: pp. 24-88).

The medicament of the present invention may contain the peptide derivative of an aryl diester of alpha-aminoalkylphosphonic acid. As the inhibitors against many serine proteases including bovine thrombin, human factor XIIa, human factor Xa, human kallikrein, bovine trypsin, rat skin tryptase, human leukocyte elastase, porcine pancreatic elastase, bovine chymotrypsin, human leukocyte cathepsin G, rat mast cell protease II, these alpha-aminoalkylphosphonate derivatives have been found (U.S. Pat. No. 5,543,396; U.S. Pat. No. 5,686,419; U.S. Pat. No. 5,952,307; and the others). These derivatives are contained in human blood plasma, and very stable under a variety of circumstances. For example, the preferred inhibitor Suc-Val-Pro-Phe^{P}(OPh)₂, particularly preferably, Suc-Val-Pro-L-Phe^{P}(OPh)₂ contained in the medicament of the present invention, has a half-life of about 20 hours in human blood plasma. This stability is important, because it is expected that a peritoneal exudate neutralizes most of protease inhibitors to damage their activity. Aminoalkylphophonic acid is analogous to an a-amino acid, and designated by the generally acceptable three-letter abbreviation for an amino acid followed by the superscript P. For example, Cbz-Ala^{P}(OPh)₂ is the abbreviation, in respect to alanine, for diphenyl alpha-(N-benzyloxycarbonylamino) ethylphosphonate.

The peptide Val^{P}(OPh)₂ having a phosphate residue at the C-terminal, which is a valin analogue, is comparatively effective and comparatively specific irreversible inhibitor specific for elastase and an elastase-like enzyme. The peptide with a phosphate residue at the C-terminal which is related with phenylallanine, the other aromatic amino acid or a long aliphatic side chain is comparatively effective and a comparatively effective inhibitor against chymotrypsin and chymotrypsin-like enzyme. The Peptide which is related with the C-terminal diphenyl ester of ortinine, lysine, arginine or alpha-amino-alpha-(4-amidinophenyl)methanephosphonate[(4-AmPhGly)^{P}(OPh)₂] or alpha-amino-alpha-(4-amizinophenymethyl) methanephosphonate [(4-AmPhe)^{P}(OPh) 2] are comparatively effective and comparatively stable inhibitors of against trypsin and trypsin-like enzymes.

Additional specificity and/or increased activation toward reaction with the enzyme can be introduced into the inhibitor molecule by way of variation in the amino acid sequence of the peptide structure site. There is generally a coincidence in sequence between an enzyme substrate such as a peptidyl p-nitroanlides and an effective peptidyl phosphonate inhibitor. Inhibitors with relatively high activity generally have the sequence of a favorable peptidyl p-nitroanilide substrate for a particular enzyme. For example a relatively potent inhibitor for chymotrypsin and chymotrypsin-like enzymes is Suc-Val-Pro-Phe^{P}(OPh)₂ which has an amino acid sequence that is analogous to Suc-Val-Pro-Phe-NA, a suitable substrate for these enzymes. With human leukocyte elastase, two relatively potent inhibitors [Meo-Suc-Ala-Ala-Pro-Val^{P}(OPh)₂ and Boc-Val-Pro-Val^{P}(OPh)₂] have an amino acid sequence similar to MeO-Suc-Ala-Ala-Pro-Val-NA and Boc-Val-Pro-Val-NA, two suitable substrates for this enzyme. It is also possible to design relatively potent phosphonate inhibitors for serine proteases based on the peptide sequences found in other relatively potent reversible and irreversible inhibitors for those same serine proteases reported in the literature (Powers and Harper, in Proteinase Inhibitors, Barrett and Salvesen, eds., Elsevier, pp. 55-152, 1986; Trainore, D. A., Trends in Pharm. Sci. 8: pp. 303-307, 1987).

Diphenyl esters of alpha-aminoalkylphosphonate can be synthesized by previously described method (U.S. Pat. No. 5,543,396). Di (substituted phenyl) esters of alpha-aminoalkylphosphonate can be also prepared by the same procedure using tris (substituted phenyl) phosphite instead of triphenyl phophite. Perfluoroalkyl diesters can be synthesized by a method involving transesterification (Szewczyket al, Synthesis, pp. 409-7414, 1982). Alternatively, the synthesis of diesters of alpha-aminoalkylphosphonic acids and their peptides can be performed by esterification of the phosphonic acid moiety as described previously (Bartlett et al., Bioorg. Chem., 14: pp. 356-377, 1986). Numerous additional serine protease inhibitors that may be synthesized for use in the present invention have been disclosed in U.S. (U.S. Pat. No. 6,262,069, U.S. Pat. No. 5,916,888, U.S. Pat. No. 5,900,400, U.S. Pat. No. 5,157,019, U.S. Pat. No. 4,829,052, U.S. Pat. No. 5,723,316, U.S. Pat. No. 5,807,829).

Many organic compounds exist in optically active forms. In describing an optically active compound, the prefixes D and L or R and S are used to denote the absolute configuration of the molecule about its chiral center(s) The prefixes (+) and (−) or d and 1 are employed to designate the sign of rotation of plane-polarized light by the compound, with (−) or 1 meaning that the compound is levorotarory and a compound prefixed with (+) or d is dextrorotatory. For a given chemical structure, these compounds, called stereoisomers, are identical except that they are mirror images of one another. A specific steroisomer may be also referred to as an enantiomer, and a mixture of such isomers may be called an enantiomeric mixture or racemic. The present invention can utilize stereochemical or optical purity as means for greater potency and/or decreased deleterious effects.

As used herein, the term “chiral” refers to molecules which have the property of non-superimposability of the mirror image partner, while the term “achiral” refers to molecules which are superimposable on their mirror image partner. The term “stereoisomers” refers to compounds which have identical chemical constitution, but differ with regard to the arrangement of the atoms or groups in space. In particular, “enantiomers” refer to two steroisomers of a compound, which are non-superimposable mirror images of one another.

“Diasteromers”, on the other hand, refers to stereoisomers with two or more centers of dissymmetry and whose molecules are not mirror images of one another. With respect to the nomenclature of a chiral center, the terms “S” and “R” configurations are as defined by the IUPAC 1974. Recommendations for Section E., Fundamental Stereochemistry, Pure Appl. Chem., 45: pp. 13-30 (1976).

The terms “enantiomerically enriched” and “non-racemic”, as used interchangeably herein with reference to compounds used in methods of the present invention refer to optically enriched compositions in which one enantiomer is enriched, compared to a racemic mixture of enantiomers in their constituents. Unless otherwise specified, such terms refer to compositons in which the ratio of the desired enantiomer relative to the undesired enantiomer is greater than 1:1 by weight. For instance, an enantiomerically enriched preparation has greater than 50% by weight of the preferred enantiomer relative to the undesirable enantiomer, more preferably at least 75% by weight, and even more preferably at least 80% by weight. Of course, the enrichment can be much greater than 80% by weight, providing a “substantially enantiomerically enriched”, “substantially non-racemic”, or “substantially optically pure” preparation, which refers to preparations which have at least 85% by weight of the desired enantiomer, more preferably at least 90% by weight, and even more preferably at least 95% by weight.

Separation of enantiomers can be accomplished in several ways known in the art. For example, a racemic mixture of two enantiomers can be separated by chromatography using a chiral stationary phase (see, e.g., “Chiral Liquid Chromatography”, W. J. Lough, Ed. Chapman and Hall, New York (1989)). Enantiomers can also be separated by classical resolution techniques. For example, formation of diastereometric salts and fractional crystallization can be used to separate enantiomers. For the separation of enantiomers of carboxylic acids, the diastereometric salts can be formed by addition of enantiomerically pure chiral bases such as brucine, quinine, ephedrine, strychnine, and the like. Alternatively, diastereometric esters can be formed with enantiomerically pure chiral alcohols such as menthol, followed by separation of the diastereometric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts. In addition to separation techniques such as those described above, the active enantiomer can be synthesized by stereospecific synthesis to produce only the desired optical isomer, using methodology well known to those skilled in the art. Chiral synthesis can result in products of high enantiomeric purity. However, in some cases, the enantiomeric purity of the product is not sufficiently high. The skilled artisan will appreciate that the separation methods described above can be used to further enhance the enantiomeric purity obtained by chiral synthesis. The optical purity of the enantiomer can be determined by methods known in the art. For example, a sample of the enantiomer can be analyzed by high performance liquid chromatography on a chiral chromatographic column.

The Chymase inhibitor as a serine protease inhibitor contained in a medicament of the present invention is utilized for a method for preventing, inhibiting or treating adhesion formation in the peritoneal cavity of a warm blooded mammal, and can be administered, intravenously orally, or percutaneously to the mammal so that at least one chymase inhibitor may exist by a sufficient amount for a certain period to permit repair of the tissue.

Preferably, the chymase inhibitor which is a peptide derivative of the aryl diester of alpha-aminoalkylphosphonic acid such as Suc-Val-Pro-Phe^{P}(OPh)₂ can be utilized. More preferably, the preparation of a peptide derivative of the aryl diester of alpha-aminoalkylphosphonic acid such as Suc-Val-Pro-L-Phe^{P}(OPh)₂ which has been enantiomerically enriched can be utilized. This Suc-Val-Pro-L-Phe^{P}(OPh)₂ is preferably obtained by enriching Suc-Val-Pro-Phe^{P}(OPh)₂ enantiomerically to 50%, 80%, or more preferably 95% or more of the total weight. The warm blooded mammnal is desirably human. In order to enrich, for example, acetone-ether is used to crystallize repeatedly.

(Preparation)

The medicament of the present invention may be prepared and administered as a pharmaceutical composition comprising the above-described protease inhibitor alone of in combination with a suitable additive. The pharmaceutical composition is not particularly limited, as long as it is used for oral or non-oral administration. For example, tablet, syrup, ample for injection, freeze dried powder for injection, ointment, and wet pack may be utilized. Various types of preparations can be made in accordance with the conventional method using a known preparation additives available for those skilled in the art such as diluent and excipient.

For example, the freeze-dried powder for injection can be prepared by dissolving an effective amount of the purified protease inhibitor in a dilution solution such as a distilled water for injection, a physiological saline, or an aqueous glucose solution, and adding, if necessary, an excipient such as carboxylmethylcepharose or sodium alginic acid, a stabilizing agent such as polyethylene glycol, sodium dextran sulfate, an amino acid, or human serum albumin, a conservation agent such as benzyl alcohol, benzalkonium chloride, or phenol, a soothing agent such as glucose, calcium gluconate, or procaine hydrochloride, and a pH adjusting agent such as hydrochloric acid, acetic acid, citric acid, or sodium hydroxide to make by a conventional method. Moreover, the ampule preparation for injection can be prepared by dissolving an effective amount of the protease inhibitor in a dilution solution such as a distilled water for injection, a physiological saline, or Ringer's solution, and adding, if necessary, an auxiliary dissolving agent such as sodium salicyclate, or mannitol, a buffer such as sodium citrate, or glycerin, an isotonic agent such as glucose or invert sugar, an additive such as a stabilizing agent, a conservation agent, a soothing agent, or a pH adjusting agent, followed by sterilizing such as a conventional heat sterilization or filtration sterilization. The sterilization method should be appropriately selected because heat sterilization process can inactivate depending on the kind of the active component.

The medicament of the present invention can be made a dosage form suitable for oral or non-oral administration in a solid state such as a tablet, a granule, a capsule, or a powder, or a liquid state such as a solution, a suspension, a syrup, an emulsion, or a lemonade, an ointment, a wet pack using a pharmaceutically acceptable carrier. If necessary, an auxiliary agent, a stabilizing agent, a moistening agent, and the other conventional additive such as lactic acid, citric acid, tartaric acid, stearic acid, magnesium stearate, clay, sucrose, corn starch, talc, gelatin, agar, pectin, peanuts oil, olive oil, cacao oil, ethylene glycol may be blended in the above-described preparation.

(Dose)

The medicament of the present invention contains an effective amount of at least one protease inhibitor, and can be administered so that the protease inhibitor may be substantially retained at an effective concentration for a sufficient time to permit epithelialization in the site of adhesion formation.

In the present invention, the acceptable amount and concentration of a protease inhibitor to administer can be determined within the range from the lowest concentration for achieving the effect, that is, “an effective amount”, to the highest concentration, “a pharmacologically acceptable” concentration. A mixture of protease inhibitors can be administered intravenously, orally, or percutaneously in a dosage form or medium (physiological saline) which is suitable so that the desired effect is obtained in a site (such as abdominal, thoracic, ophthalmic, cardiac, gynecologic tissue) to prevent, inhibit or treat the adhesion formation. In the present invention, percutaneous application refers to the coating or pasting of a preparation such as an ointment or a wet pack, and is distinguished from the procedure for applying to a certain site of the surface tissue of the subject.

In the present invention, the term “effective amount” means a sufficient amount of the medicament for gaining a desired reaction with little or no toxicity to prevent, inhibit or treat the adhesion formation. The precise required amount varies depending on an individual subject in species, age, body weight, general body condition, or administration mode. A suitable “effective amount” can be also determined by the general prior art using the fact provided herein and conventional methods.

The term “pharmaceutically acceptable” refers, in consideration of effect versus danger in ratio, to a substance, a compound, a mixture or an administration mode which is appropriate to contact with human or animal tissue within a reliable medical discretion and free from accompanying with excessive toxicity, inflammation, allergic response or the other troubles.

The relevant protease inhibition compound can be administered at an interval for generally acting, for example, before, during, or after the operation. The administration can be initiated at least between 1 hour and 72 hours, and preferably between 1 hour and 48 hours after the operation. For example, in the case where it is intravenously administered, the administration can be initiated between 1 hour and 24 hours, and preferably between 1 hours and 12 hours after the operation, and in the case where it is orally administered, the administration can be initiated similarly, between 1 hour and 72 hours, and preferably between 12 hours and 48 hours, more preferably, between 12 hours and 24 hours after the operation. Furthermore, in the case where an ointment or the like is percutaneously applied, the administration can be initiated at least between 1 hour and 72 hours, preferably between 1 hour and 48 hours after the operation. Because the postoperative adhesion can be generally formed in the period from 24 hours to 14 days after the operation, the medicament of the present invention can be administered continuously within this period intravenously, orally, and percutaneously.

The medicament of the present invention allows at least one protease inhibitor to be administered in a substantially sufficient amount to permit epithelialization in the site of adhesion formation. For example, chymase inhibitor (Suc-Val-Pro-L-Phe^{P}(OPh)₂), which is a specific example of protease inhibitor contained in the medicament of the present invention, could be used at 10 μM during the operation, to suppress significantly the chymase activity in the blood vessel tissue of the living body for 4 weeks. The effective administration amount of chymase inhibitor (Suc-Val-Pro-L-Phe^{P}(OPh)₂) can be selected in the range of 0.0001 to 100 mg per 1 kg of adult body weight, per one day. For example, it can be administered intravenously by an amount selected in the range from 0.0001 to 100 mg, preferably from 0.01 to 1 mg, and orally by an amount selected in the range from 0.1 to 100 mg, preferably from 0.1 to 10 mg. Furthermore, it can be intravenously applied by an amount in the range from 0.0001 to 1000 mg, preferably from 0.1 to 100 mg.

An effective amount and pathway for the other protease inhibitors can be determined by known means.

### EXAMPLES

The present invention will be concretely described by Examples below, but is not limited to them.

### Example 1

(Preparation of alpha-aminoalkylphosphonate Derivatives)

Preparation of peptidyl derivatives of aryl diesters of alpha-aminoalkylphosphonic acids has been documented and may be performed by one of those skilled in the art (Biochemistry 30: pp. 485-493, 1991).

To elucidate the relationship between chymase activity and adhesion formation, the effect of a representative serine protease inhibitor, the chymase inhibitor, Suc-Val-Pro-Phe^{P}(OPh)₂, was tested. This compound has been synthesized using a known methodology (Biochemistry 30: pp. 485-493, 1991). More specifically, the reaction of Cbz-Val-OH (0.25 g, 1 mmol), DCC (0.2 g, 1 mmol) and the product of hydrogenolysis of Cbz-Val-Pro-Phe^{P}(OPh)₂ (0.584 g, 1 mmol) is dissolved in 30 ml of ethyl acetate and oil is added to this. To this solution, 0.1 g (1 mmol) of succinic anhydride and 0.1 g of 5% Pd/C is added and mixture is stirred under atmosphere of hydrogen unit thin layer chromatography (TLC) shows only one new spot. Catalyst is removed by filtration and the organic layer is washed several times with water. After drying, the organic solvent is removed to give, for example, 0.45 g (65%) of product as a hydroscopic solid (mp. 50-53° C.: one apot on TLC, R_{f}=0.4; ³¹P NMR 19.75, 19.23 ppm, ratio 1:1,

Anal. Calcd. for C₃₄H₄₀O₃N₃P.2H₂O: 59.56; H, 6.42. Found: C, 59.59; H, 6.42).

### Example 2

(Preparation of Enriched Enantiomers of Chymase Inhibitor Suc-Val-Pro-Phe^{P}(OPh)₂)

As used below, the following abbreviations apply: Z is Benzyloxycarbonyl, Boc is tert-butyloxycarbonyl, WSCD is Carbodiimide, and HOBt. Oleksyszyn and Powers (Methods Enzymol. 244: 423-441, 1994), and Benzyloxycarbonyl was removed by hydrogen bromide/acetic acid solution. The sample was coupled with Boc-Pro by WSCD-HOBt reaction and then the racemic mixture was obtained. The racemic mixture then was separated by re-precipitation. Inactive enantiomer was firstly crystallized from the solution and removed. After de-blocking of Boc with HCl from the active enantiomer in the solution, the sample was coupled with Boc-Val by WSCd-HOBt reaction and deblocked Boc again. To this product, succinic anhydride and triethyamine were added and Suc-Val-Pro-Phe^{P}(OPh)₂ was obtained. The product was finally enriched by reverse phase HPLC.

(Z-DL-Phe^{P}(OPh)₂)

Phenylacetaldehyde (28.3 mL, 0.242 mol) was dissolved in 45 mL of acetic acid. Carbamic acid benzyl ester (24.4 g, 0.161 mol) and triphenyl phosphite (50.0 g, 0.161 mol) were added to this solution and stirred for 1.5 hours at 85° C. After the organic solvent were evaporated and the residual solution was cooled to the room temperature, 400 mL of methanol was added to his solution and it was allowed to crystallized by friction at −20° C. The product was collected by filtration, washed with cold methanol and dried in vacuo to yield 32.9 g (42%) of mixture of Z-D-Phe^{P}(OPh)₂ and Z-L-Phe^{P}(OPh)₂.

(DL-Phe^{P}(OPh)₂ HBr)

The mixture of Z-Phe^{P}(OPh)₂ and Z-L-Phe^{P}(OPh)₂ (14.3 g, 29.3 mmol) was dissolved in 30 mL of 25% hydrogen bromide/acetic acid solution and stirred for 1 hour at room temperature. After addition of ether, the separated solid sample was collected by filtration. This sample was washed with ether and dried in vacuo to yield 12 g (94%) of the mixture of D and L-Phe^{P}(OPh)₂—HBr.

(Boc-Pro-DL-Phe^{P}(OPh)₂)

The mixture of D and L-Phe^{P}(OPh)₂.HBr (11.5 g, 26 mmol), Boc-Pro (5.53 g, 25.7 mmol) and HOBt (3.58 g, 26.5 mmol) were dissolved in 70 mL of DMF and WSCD (4.70 mL, 26.5 mmol) was added drop-wise into this solution under cooled with ice. After being stirred for 3.5 hours at room temperature, the solution was concentrated in vacuo and ethyl acetate was added. The resulting solution was successively washed with acidic and alkaline solution, and dried over MgSO₄, and concentrated in vacuo. The inactive enantiomer (D) was crystallized from acetone-ether and removed, then Boc-Pro-L-Phe^{P}(OPh)₂ was obtained. The residual solution was concentrated in vacuo and purified by medium-pressure silica-gel with toluene-ethyl acetate (5:1) as eluent to yield 5.10 g of the enantiomer.

(Boc-Pro-L-Phe^{P}(OPh)₂)

Boc-Pro-L-Phe^{P}(OPh)₂ (4,98 g, 9.05 mmol) was dissolved with 37 mL of cold HCl/dioxane (4.9 N) and stirred for one hour at room temperature. The solution was evaporated and dried in vacuo. The residue was dissolved with 40 mL of DMF, and Boc-Val (2.06 g, 9.50 mmol) and HOBt (1.35g, 9.96 mmol) were added to this solution. WSCD (1.77 mL, 9.96 mmol) was added dropwise into this solution under cooled with ice and stirred over night at room temperature. Ethyl acetate was added to this reacting solution and successively washed with acidic and alkaline solution. After being dried over MgSO₄ and filtered, solution was concentrated in vacuo to yield 6.26 g (94%) of product, as a colorless oil.

(Suc-Val-Pro-Phe^{P}(OPh)₂)

Boc-Val-Pro-Phe^{P}(OPh)₂ (5.86 g, 8.47 mmol) was dissolved with 35 mL of cold HCl/dioxane (4.9 N) and stirred for one hour at room temperature. The solution was evaporated and dried in vacuo. The residue was dissolved with 35 mL of DMF, and succinic anhydride (1.02 g, 10.2 mmol) was added. Triethylamine (2.36 mL, 16.9 mmol) was added drop-wise into this solution and stirred for two hours at room temperature. Under cooled with ice, pH of solution was adjusted to pH 1.0 by HCl and the sample was extracted with ethyl acetate. The extract was washed with saturated saline solution, dried over MgSO₄, filtered and concentrated in vacuo to yield the product, as a yellow oil. The resulting oil was enriched by the reverse-phase HPLC (Column: YMC ODS SH-363-5, 30×250 mm, Mobile phase: 0.1% TFA gradient 40-70% MeCN) and freeze-dried to yield 2.30 g (42%) of product as a white powder.

(Results of Analysis)

Sample: Suc-Val-Pro-Phe^{P}(OPh)₂

Lot.: 520217

Volume: 2.0 g×1

Appearance: White powder

Purity (by HPLC): Main peak 98.8%

HPLC conditions

Column: YMC Pack, ODS-A 4.6 mm I.D.×150 mm

Mobile phase: 0.1% TFA, gradient 30-80% MeCN (25 min)

Flow rate: 1.0 mL/min

Detector: An ultraviolet absorption photometer (wavelength: 220 nm)

| Amino acid analysis: | molar ratio | recovery |
|---|---|---|
| Val (l) | 1.00 | 90.5% |
| Pro (l) | 1.03 | |
| Phe^{P}(OH)₂ | 0.98 | |

Hydrolysis conditions; 6 mol HCl with phenol, 110° C., 22 hours

Elemental analysis: Calcd for C₃₄H₄₀N₃O₈S: C, 62.86: H, 6.21; N, 6.47%

Calcd for C₃₄H₄₀N₃O₈S.8H₂O.0.5 TFA: C, 58.30: H, 5.88; N, 5.83% Hound: C, 58.34; H, 5.94; N, 5.51%

Mass spectrometry: 650.3 (Calcd [M+H]_{extract}=650.253 (by ESI-MS))

### Experimental Example

### Prevention Effect of Adhesion by Oral Administration of Suc-Val-Pro-Phe^{P}(OPh)₂)

Syrian hamsters (SLC Co., Ltd.), 6 weeks of age (weighing 85-90 g), were subject to abdominal middle incision to scrape their hearts. Cardiac muscle adhesion models were prepared. Suc-Val-Pro-Phe^{P}(OPh)₂ (10 mg/kg) (eight cases) or placebo (eight cases) was forcibly orally administered using a canal once a day from the 3rd day before the model preparation to the 14th day after the model preparation. The adhesion levels at the 14th day after the model preparation were compared to study. Adhesion scores are as follows. Student-t test was used to test a significant difference in adhesion core.

Adhesion level 0: A heart is easily taken out, and the heart exists independently from the circumference, as is similar to a normal heart.

Adhesion level 1: A heart is observed to adhere to the circumferential tissue at one site by a thin film, but the adhesion site can be simply freed to release the heart.

Adhesion level 2: A heart is observed to adhere to the circumferential tissue at a few sites, but the adhesion sites can be freed by a traction force to release the heart.

Adhesion level 3: A heart is firmly adhered, but is distinguishable from, to the circumferential tissue at several sites, and it is hard to release the heart without a strong traction force.

Adhesion level 4: Excessive cell growth and fibrosis occur around a heart, and the heart is observed to adhere to the circumferential tissue. The heart is so firmly adhered to the circumferential tissue that the heart is difficult to release even by a peeling traction force.

In the placebo group, there were three cases (37.5%) ranked as adhesion level 4, two cases (25%) as adhesion level 3, one case (12.5%) as adhesion level 1, and the remaining cases were ranked as adhesion level 0 (25%). In the Suc-Val-Pro-Phe^{P}(OPh)₂ administration group, there were one case (12.5%) ranked as adhesion level 4, one case (12.5%) as adhesion level 2, and the remaining cases were ranked as adhesion level 0 (75%), indicating that the group fell down significantly in adhesion level.

### INDUSTRIAL APPLICABILITY

As described above, the medicament of the present invention for preventing, inhibiting or treating adhesion formation is administered intravenously, orally, or percutaneously to be useful for preventing adhesion, and to be useful against not only the postoperative adhesion of orthopedic or plastic surgery but also inflammatory visceral adhesion.

1. A medicament for preventing, inhibiting, or treating adhesion formation of the tissue surface within a vertebrate subject, wherein the medicament contains an effective amount of at least one protease inhibitor and is administered intravenously, orally, or percutaneously.

2. The medicament for preventing, inhibiting or treating adhesion formation according to claim 1, wherein the protease inhibitor is a serine protease inhibitor.

3. The medicament for preventing, inhibiting or treating adhesion formation according to claim 2, wherein the serine protease inhibitor is a chymotrypsin-like serine protease inhibitor.

4. The medicament for preventing, inhibiting or treating adhesion formation according to claim 3, wherein the chymotrypsin-like serine protease inhibitor is a chymase inhibitor.

5. The medicament for preventing, inhibiting or treating adhesion formation according to claim 4, in which the relevant chymase inhibitor is a peptide derivative of aryl diester of alpha-aminoalkylphosphonic acid.

6. The medicament for preventing, inhibiting or treating adhesion formation according to claim 4, wherein the chymase inhibitor is Suc-Val-Pro-Phe^{P}(OPh)₂.

7. The medicament for preventing, inhibiting or treating adhesion formation according to claim 4, wherein the chymase inhibitor is a concentrated preparation of enantiomer Suc-Val-Pro-L-Phe^{P}(OPh)₂ of Suc-Val-Pro-Phe^{P}(OPh)₂.

8. The medicament for preventing, inhibiting or treating adhesion formation according to claim 7, wherein Suc-Val-Pro-L-Phe^{P}(OPh)₂ contains 95% or more of the total weight of Suc-Val-Pro-Phe^{P}(OPh)₂ in the concentrated preparation of the enantiomer.

9. The medicament for preventing, inhibiting or treating adhesion formation according to claim 1, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

10. The medicament for preventing, inhibiting or treating adhesion formation, wherein the medicament comprises the protease inhibitor according to claim 1 and a pharmaceutically acceptable diluent solution or excipient.

11. A method for preventing, inhibiting or treating adhesion formation, wherein the medicament for preventing, inhibiting or treating adhesion formation according to claim 1 is administered to a vertebrate subject before surgical operation, during the surgical operation, after the surgical operation, or in the case of possible inflammatory visceral adhesion.

12. The medicament for preventing, inhibiting or treating adhesion formation according to claim 2, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

13. The medicament for preventing, inhibiting or treating adhesion formation according to claim 3, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

14. The medicament for preventing, inhibiting or treating adhesion formation according to claim 4, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

15. The medicament for preventing, inhibiting or treating adhesion formation according to claim 5, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

16. The medicament for preventing, inhibiting or treating adhesion formation according to claim 6, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

17. The medicament for preventing, inhibiting or treating adhesion formation according to claim 7, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

18. The medicament for preventing, inhibiting or treating adhesion formation according to claim 8, wherein the protease inhibitor is bound to a transmitter for maintaining an effective local concentration of the protease inhibitor in the relevant site and then administered, the transmitter being a carrier having a high molecular weight selected from the group consisting of hyaluronic acid, hydrogel, carboxymethylcellulose, dextran, cyclodextran and a composition of compounds thereof.

19. The medicament for preventing, inhibiting or treating adhesion formation, wherein the medicament comprises the protease inhibitor according to claim 2, and a pharmaceutically acceptable diluent solution or excipient.

20. The medicament for preventing, inhibiting or treating adhesion formation, wherein the medicament comprises the protease inhibitor according to claim 9, and a pharmaceutically acceptable diluent solution or excipient.
